(19) United States
(12) Patent Application Publication
(10) Pub. No.: US 2025/0082859 A1
(43) Pub. Date: Mar. 13, 2025

(54) Needle

(71) Applicant: SEFT HOLDING, SION (CH)

(72) Inventor: Jean Pascal ZAMBAUX, BOULIAC (FR)

(21) Appl. No.: 18/887,311

(22) Filed: Sep. 17, 2024

(30) Foreign Application Priority Data
Sep. 19, 2023 (FR) FR 2309922

(51) Int. Cl. A61M 5/32; A61B 17/00

(52) U.S. Cl. CPC A61M 5/32; A61B 2017/00867

(57) ABSTRACT

The invention relates to a needle (1) comprising a cylindrical body (10) extending along a longitudinal axis, at least one end (100) of which is beveled, said body (10) being formed at least from a polymer, said needle (1) being characterized in that it comprises at least one reinforcing thread (101) adapted to stiffen said body (10), said reinforcing thread (101) being formed at least from a shape memory material.

### TECHNICAL FIELD

The invention relates to the technical field of needles. The invention relates in particular to a syringe comprising said needle.

### PRIOR ART

Pyrogens or pyrogenic substances are impurities which, when present in a pharmaceutical product intended for administration to a patient, cause a febrile reaction when the pharmaceutical product is actually administered. In the medical field, many devices feature injection and/or perforation needles for the flow of a pharmaceutical product, usually a liquid. In addition to being sterile and sterilizable, such needles intended to contain and transport a pharmaceutical product must also be pyrogen-free, that is they must not contain pyrogens, and must be depyrogenizable, that is they must be able to undergo a treatment that eliminates any pyrogens they may contain. To depyrogenate such needles, they have to be heated to 253° C. for an hour. This is one of the reasons why needles for injecting pharmaceutical products into human tissue are generally made of metal. Another reason is that these needles for injection into human tissue must be very thin to limit the pain felt by the patient, while retaining sufficient strength and rigidity to penetrate the skin and subcutaneous layers. However, once used, metal needles cannot be easily disposed of, which poses a health problem due to contamination accidents resulting from contact with these unsterilized and non-depyrogenated needles. To solve the problem of needles for injecting pharmaceutical products, particularly into the human body, plastic needles have been proposed.

In reality, when a plastic needle for medical use is used, it is single-use and not depyrogenated, but rather manufactured under conditions such that it is pyrogen-free. However, once out of its packaging and in use, it cannot be depyrogenated. What's more, as these needles cannot be depyrogenated, it is not possible to depyrogenate them before first use, to be sure that they are indeed apyrogenic. In fact, production problems or problems during transport or handling may have led to contamination by pyrogens.

In particular, we know of needles which may be mounted on an injection syringe, and which are both depyrogenizable and easily disposable. In particular, this type of needle may consist of a cylindrical body extending along a longitudinal axis, at least one end of which is beveled and made of a polyaryletherketone polymer reinforced with stainless steel threads. However, one disadvantage of this type of needle is that they are rigid and not very adaptable.

The invention therefore takes place in this context and seeks to solve all of the aforementioned drawbacks. The aim of the invention is to provide a needle that avoids needlestick injuries by being stainless steel-free, but with a rigid needle body.

### Overview of the Invention

The object of the invention is a needle comprising a cylindrical body extending along a longitudinal axis, at least one end of which is beveled, said body being formed at least from a polymer, said needle being characterized in that it comprises at least one reinforcing thread adapted to stiffen said body, said reinforcing thread being formed at least from a shape-memory material.

The cylindrical body may be hollow. The hollow body may have a circular cross-section. The hollow body may provide a conduit for the flow, preferably of a liquid, in particular a pharmaceutical liquid to be injected or transfused.

The needle body may have at least one beveled end that may be used to perforate human tissue in particular. In an embodiment wherein the needle has a single beveled end, the other end may be adapted for insertion into a syringe.

In one embodiment, the body may comprise at least one reinforcing thread embedded in the polymer of said body. The thread may extend parallel to the longitudinal axis along the entire length of the hollow body. In an embodiment comprising several reinforcing threads, the body may comprise three reinforcing threads embedded in the polyaryletherketone polymer, extending parallel to the longitudinal axis X-X′ over the entire length of the hollow body, being equally tensioned and equidistant from one another and distributed so that the threads in pairs define an identical angle at the center. The three reinforcing threads may be tensioned evenly along their entire length.

The needle may be adapted for injection into human tissue and may have an external diameter of around 0.7 mm and an internal diameter of around 0.25 mm, which means that the needle wall thickness may be around 0.25 mm. At such wall thicknesses, rigidity, that is resistance to deformation, may be insufficient. The needle may have a small thickness as mentioned above to help minimize pain and tissue damage for the patient.

The polymer may be polyaryl ether ketone, polyethylene, polycarbonate or polypropylene. The polymer may comprise fillers selected from carbon fibers, glass fibers, graphite granules, polytetrafluoroethylene (PTFE) granules, black carbon granules and mixtures of two or more thereof.

The thread may have a diameter of less than 500 μm, in particular less than 300 μm. In view of this diameter, when the needle is exposed to flame or high-temperature heating, in order to render it unusable and avoid the possibility of perforation from its beveled end, the threads can also melt and the needle's health safety may be maintained.

The shape-memory material may be soft and/or elastic for a first temperature range and may be rigid for a second temperature range.

Advantageously, the polymer is an oxy-1,4-phenylene-oxy-1,4-phenylene-carbonyl-1,4-phenylene.

The polymer may be the one marketed under the brand name PEEK® (registered trademark). The polymer may be a semi-crystalline aromatic linear polymer. The polymer may have a melting temperature of 343°. The polymer may have a glass transition temperature of 143° C. The polymer may have an operating temperature of about 260° C. The polymer may have a softening temperature of up to 315° C. The needle comprising the polymer may be depyrogenable, that is it may be heated to about 253° C. for notably one hour without undergoing deformation or alteration of its chemical and mechanical properties. The polymer may be adapted to support a subsequent extrusion step. The polymer's coefficient of expansion is roughly the same as that of stainless steel, so it may be solidified by heating to stiffen, and retains the same shape after stiffening.

The polymer may be biocompatible with human tissue. The polymer may be intrinsically pure, with very low levels of gas release and extractable ions.

Advantageously, the reinforcing thread has at least one spiral-shaped portion around said longitudinal axis.

The reinforcing thread may be embedded in the polyaryletherketone polymer, extending around an axis of rotation coincident with the longitudinal axis along the entire length of the hollow body. In the embodiment comprising three threads, said threads may be arranged so that each extends around an axis of rotation coincident with the longitudinal axis over the entire length of the hollow body, said threads having an identical direction of rotation. The three threads may be equally spaced from one another.

Advantageously, the needle comprises a plurality of reinforcing threads forming a mesh.

In this embodiment, the plurality of threads extends around the longitudinal axis along the entire length of the hollow body, so that said threads intertwine to form a mesh. The threads may overlap and cross.

Advantageously, the thread comprises a stiffened portion and a flexible portion.

In other words, the thread may be made of the same material over its entire length, but with a stiffened part and a non-stiffened part. In another embodiment, the thread may comprise two materials, a first material forming a stiffened portion and a second material forming a non-stiffened portion. A needle may thus comprise a flexible portion and a rigid or semi-rigid portion.

Advantageously, each thread is circular in cross-section.

In this embodiment, each thread may have a diameter of around 0.1 mm.

Advantageously, each thread is elliptical in cross-section.

In this embodiment, each thread may have a main axis of around 0.15 mm.

Advantageously, each thread is triangular in cross-section.

The triangular cross-section thread may be advantageously used, since it may be stiffer than circular-section thread.

Advantageously, the reinforcing thread is arranged inside the needle body.

In another embodiment, the thread may form a shape-memory cylinder, that is the thread may form a hollow cylinder positioned around the needle and within the lumen of the hollow body. In this embodiment, the needle may be disposable and the shape-memory cylinder reusable, so the stiffened shape-memory part avoids contact with the product.

Advantageously, the body of said needle is obtained using a method comprising a first step of extruding the polyaryletherketone polymer simultaneously with a step of pultruding the at least one reinforcing thread within said extruded polyaryletherketone polymer.

The polymer extrusion may be simultaneous with the insertion of the thread into the extruded channel. The pultrusion step may correspond to a thread insertion step.

Advantageously, said at least one reinforcing thread is stiffened simultaneously with the pultrusion step.

The reinforcing thread may stiffen at temperatures above 80°. In one embodiment, the thread may stiffen at a temperature suitable for sterilization.

Stiffening of the reinforcing thread may be achieved by heating the thread. Once the thread has reached its stiffening temperature, it may take on its shape and, on cooling, retain this shape.

Once the thread is stiffened, sterilization has no impact on the rigidity of the body.

In one embodiment, the needle body is obtained by a method comprising a first step of extruding the polyaryletherketone polymer to form a body provided with a channel and a second step of inserting the at least one thread into said channel of the body. In this embodiment, the thread may be inserted at the end of the extrusion method to prevent the polymer from melting when the thread is heated, thus preventing the thread from coming into contact with the lumen of the needle body and preventing it from contaminating the products to be injected.

In another embodiment, the at least one reinforcing thread is stiffened, subsequent to the pultrusion step, in a dry heat sterilization step wherein the body of said needle incorporating the reinforcing thread is heated to a temperature of at least 160°.

Advantageously, said at least one reinforcing thread is stiffened, subsequent to the pultrusion step, in a wet heat sterilization step wherein the body of said needle incorporating the reinforcing thread is heated to a temperature of at least 121°.

The reinforcing thread may be heated using either dry or wet heat.

Advantageously, the body of said needle is obtained using a method comprising a first step of additive printing of the body from a spool of polyaryletherketone polymer thread around the at least one reinforcing thread.

The cylindrical body may comprise a circular cross-section that may help minimize pain when the needle is inserted into human tissue.

Advantageously, the body of said needle is obtained by a method comprising a first step of subtractive printing of the body in a block of polyaryletherketone polymer wherein the at least one reinforcing thread is arranged.

Advantageously, the reinforcing thread is arranged inside the needle body.

In this embodiment, the thread may form a sheath with a lumen, and the polymer forming the needle body may be inserted into said lumen.

Advantageously, the body of said needle is obtained according to a first method, and the reinforcing thread is obtained according to a second method to form a sheath inside which the needle body is arranged.

Advantageously, the at least one thread is made of composite material.

The material may be a shape-memory alloy (SMA), particularly copper-based (e.g. chemical formula CuAlBe, CuAlNi, CuAlMn, CuZnAl), nickel-titanium-based (e.g. chemical formula NiTi, NiTiFe, NiTiCu, NiTiCr, NiTiHf, NiTiCo, NiTiNb etc.), and iron-based alloys (e.g. chemical formula FeMnSi, FeMnCr, FeMnCrSi). Some of these shape-memory alloys may be monocrystalline, that is made up of a single grain, or of several grains separated by low-disorientation grain boundaries.

The activation temperature of the composite material during heating, that is in the transition from the martensite phase to the austenite phase, may be between 45° C. and 450° C.

Another aspect of the invention relates to an injection syringe comprising a plunger; a pump body equipped with a terminal fitting for fitting an injection needle; an injection needle as previously described.

The syringe may be adapted for injecting pharmaceutical products, particularly into human tissue. The syringe may comprise a pump body acting as a reservoir for a pharmaceutical product to be injected. The pump body may comprise an end fitting with a tip adapted to receive the injection needle. The pump body may comprise a plunger arranged in said pump body, adapted to propel the pharmaceutical product through the needle.

The needle and syringe of the invention may be produced by any method known to the person skilled in the art, such as an injection molding method, an extrusion method or a tension extrusion method known as a pultrusion method, wherein the reinforcing threads are held under tension as they and the polymer matrix pass through the extrusion die.

The needle, syringe and connector of the invention may be produced by any method known to the person skilled in the art, such as an injection molding method, an extrusion method or a tension extrusion method known as a pultrusion method, wherein the reinforcing threads are held under tension as they pass through the extrusion die.

Advantageously, the pump body with its end fitting and the plunger are made of a polyaryletherketone polymer.

The syringe may be made of a polyaryletherketone polymer, which means that the plunger and the pump body with its tip may be made entirely of such a polymer.

Advantageously, the pump body equipped with its end fitting and the plunger are made of a polyaryletherketone polymer comprising fillers selected from glass fibers, carbon fibers, graphite granules, polytetrafluoroethylene (PTFE) granules, carbon black granules and mixtures of two or more of these.

### BRIEF DESCRIPTION OF THE FIGURES

Other advantages and features of the present invention are now described with the aid of an example that is purely illustrative and in no way limiting as to the scope of the invention, and based on the attached drawings, wherein the various figures represent:

FIG. 1 shows a partial view in longitudinal section of the end of a needle according to one embodiment of the invention.

FIG. 2 shows a partial longitudinal view of the end of a needle according to another embodiment of the invention.

FIG. 3 shows a partial longitudinal view of the end of a needle according to a different embodiment of the invention.

FIG. 4 shows a cross-sectional view along a needle according to one embodiment.

FIG. 5 shows a partial longitudinal section view of a syringe with a needle according to the invention.

In the following description, identical elements, by structure or function, appearing in different figures retain, unless otherwise specified, the same references.

### DESCRIPTION OF ONE EMBODIMENT

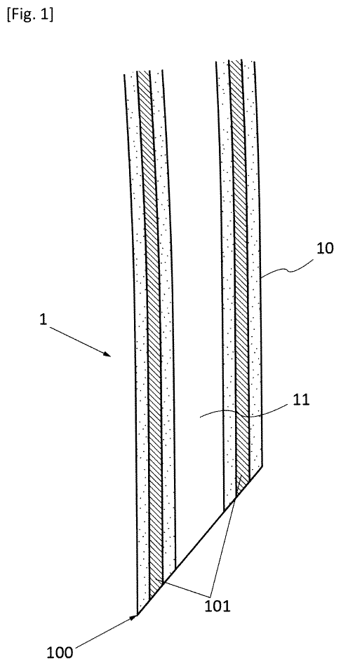

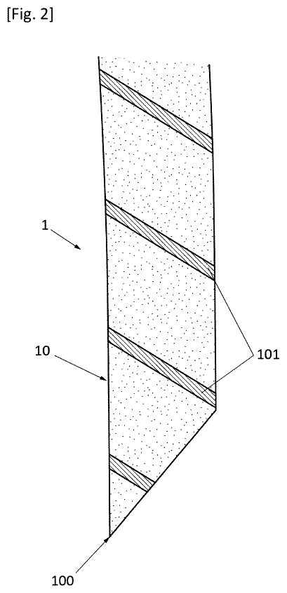

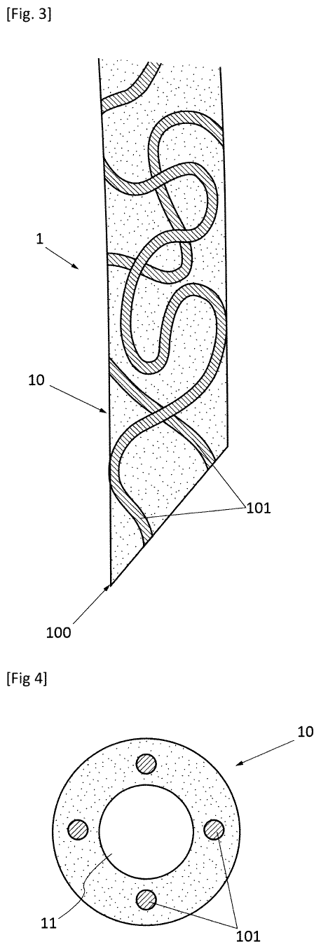

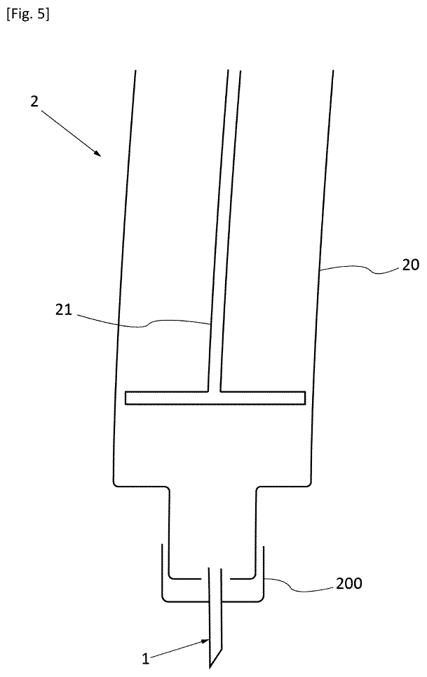

FIG. 1 shows a partial view in longitudinal section of the end of a needle according to one embodiment of the invention. The needle is also described in connection with FIG. 2 and FIG. 3 each representing different embodiments of the needle and with FIG. 4 representing a cross-sectional view of a needle according to one embodiment. The needle is also described in FIG. 5, in an embodiment wherein the needle is used in a syringe.

The needle 1 comprises a cylindrical body 10 extending along a longitudinal axis. The needle 1 has at least one beveled end 100. The beveled end 100 is suitable for perforating human tissue. The cylindrical body 10 is hollow and has a central lumen 11. The body 10 has a circular cross-section. The body 10 comprises at least one polymer. The polymer is an oxy-1,4-phenylene-oxy-1,4-phenylene-carbonyl-1,4-phenylene. The polymer solidifies on heating to become rigid and retains the same shape after rigidification.

The needle 1 comprises at least one reinforcing thread 101 adapted to stiffen the body. The reinforcing thread 101 comprises a shape-memory material. The material may be a shape-memory alloy. The reinforcing thread is embedded in the body polymer. Each reinforcing thread has a diameter of 0.1 mm.

In the embodiment described in FIG. 1, the body 10 comprises three threads 101 extending parallel to the longitudinal axis along the entire length of body 10. The threads 101 are equidistant from each other and distributed so that the threads 101 in pairs define an identical center angle.

In the embodiment described in FIG. 2, the body 10 comprises four threads 101 extending around the body 10 of the needle 1. The reinforcing threads 101 have at least one spiral-shaped portion extending around the longitudinal axis of the body. The threads 101 have an identical direction of rotation. The threads 101 are equally spaced from each other. The reinforcing thread 101 may be embedded in the polymer, extending around an axis of rotation coincident with the longitudinal axis along the entire length of the body 10.

In the embodiment described in FIG. 3, the body 10 comprises a plurality of threads 101 forming a mesh. The plurality of threads 101 extends around the longitudinal axis along the entire length of the body 10, so that said threads 101 interweave to form a mesh. The threads 101 overlap and intersect.

In the embodiment described in FIG. 4, each thread 101 has a circular cross-section and the needle 1 has four reinforcing threads 101.

FIG. 5 shows an injection syringe 2 with a plunger 21 and a pump body 20 fitted with needle 1.

The pump body 20 acts as a reservoir for a pharmaceutical product to be injected. The pump body 20 may comprise an end fitting 200 with a tip adapted to receive the injection needle 1. The pump body 20 may comprise a plunger 21 arranged in said pump body 20, adapted to propel the pharmaceutical product through the needle 1.

The pump body 20 with its end fitting 200 and the plunger 21 are made of polymer. The pump body 20, end fitting 200 and plunger 21 are made of polyaryletherketone polymer.

The preceding description clearly explains how the invention makes it possible to achieve the stated objectives, namely to propose a needle making it possible to avoid needleprick injuries by being devoid of stainless steel but whose needle body is rigid, by proposing a needle comprising a cylindrical body extending along a longitudinal axis, at least one end of which is beveled, said body being formed at least from a polyaryletherketone polymer, said needle being characterized in that it comprises at least one reinforcing thread adapted to stiffen said body, said reinforcing thread being formed at least from a shape-memory material.

In any case, the invention is not limited to the embodiments specifically described in this document, and extends in particular to any equivalent means and to any technically operative combination of these means. It will in particular be possible to envisage that:
Each thread is elliptical in cross-section;
Each thread is triangular in cross-section;
The cylindrical body is beveled at both ends;
The reinforcing thread is arranged inside the needle body;
The thread may form a shape-memory cylinder, that is the thread may form a hollow cylinder positioned around the needle and within the lumen of the hollow body;

The thread may be arranged around the needle body to form a sheath with a lumen, and the polymer forming the needle body may be inserted into said lumen.

1. A needle (1) comprising a cylindrical body (10) extending along a longitudinal axis, at least one end (100) of which is beveled, said body (10) being formed at least from a polymer, said needle (1) being characterized in that it comprises at least one reinforcing thread (101) adapted to stiffen said body (10), said reinforcing thread (101) being formed at least from a shape-memory material.

2. The needle (1) according to claim 1, characterized in that the polymer is oxy-1,4-phenylene-oxy-1,4-phenylene-carbonyl-1,4-phenylene.

3. The needle (1) according to claim 1, characterized in that the reinforcing thread (101) has at least one portion in the form of a spiral about said longitudinal axis.

4. The needle (1) according to claim 1, characterized in that it comprises a plurality of reinforcing threads (101) forming a mesh.

5. The needle (1) according to claim 1, characterized in that the thread (101) comprises a rigidified portion and a flexible portion.

6. The needle (1) according to claim 1, characterized in that the reinforcing thread (101) is arranged inside the needle body (10).

7. The needle (1) according to claim 6, characterized in that the body (10) of said needle (1) is obtained according to a method comprising a first step of extruding the polyaryletherketone polymer simultaneously with a step of pultruding at least one reinforcing thread (101) within said extruded polyaryletherketone polymer.

8. The needle (1) according to claim 6, characterized in that the body of said needle (1) is obtained according to a method comprising a first step of additive printing of the body (10) from a spool of polyaryletherketone polymer thread around the at least one reinforcing thread (101).

9. The needle (1) according to claim 6, characterized in that the body (10) of said needle (1) is obtained by a method comprising a first step of subtractive printing of the body (10) in a polyaryletherketone polymer block wherein the at least one reinforcing thread (101) is arranged.

10. An injection syringe (2) comprising:
A plunger (21);
A pump body (20) equipped with an end fitting (200) for adjusting an injection needle (1);
An injection needle (1) according to any of the preceding claims.
